(19) United States
(12) Patent Application Publication
(10) Pub. No.: US 2004/0209959 A1
(43) Pub. Date: Oct. 21, 2004

(54) Congeners of acetaminophen and related compounds as substrates for fatty acid conjugation and their use in treatment of pain, fever and inflammation

(76) Inventors: Edward Hogestatt, Lund (SE); Peter Zygmunt, Lund (SE)

Correspondence Address: BIRCH STEWART KOLASCH & BIRCH, PO BOX 747, FALLS CHURCH, VA, 22040-0747, US

(21) Appl. No.: 10/484,071

(22) PCT Filed: Jul. 16, 2002
(86) PCT No.: PCT/SE02/01392

Related U.S. Application Data
(60) Provisional application No. 60/305,160, filed on Jul. 16, 2001.

(30) Foreign Application Priority Data
Jul. 16, 2001 (SE) 0102548-5

(51) Int. Cl. A61K 31/16

(52) U.S. Cl. 514/625; 514/629

(57) ABSTRACT

The present invention relates to new analgesic, antipyretic and/or anti-inflammatory compounds represented by the general formula X—Y, in which X is a benzyl group, a saturated or unsaturated cycloalkyl group (I,II) or a non-cyclic, straight or branched alkyl group (III,IV).

### FIELD OF THE INVENTION

The present invention relates to new compounds undergoing for fatty acid conjugation in vivo and their use in treatment of pain, fever and inflammatory conditions.

### BACKGROUND OF THE INVENTION

The enzyme cyclo-oxygenase (COX) is the main target for existing light analgesics and non-steroidal anti-inflammatory drugs (NSADDs) (22). To date, two isoforms of COX have been identified—COX-1 and COX-2. They are key enzymes in the production of prostaglandins, which are mediators of fever, pain and inflammation (22). COX is widely distributed throughout the body. As a result, drugs (e.g. acetylsalicylic acid) targeting COX have a number of side effects such as gastro-intestinal ulcerations and bleedings (22). The opioids dextropropoxiphen, codeine and tramadole are other common light analgesics. The drawback with these drugs is, however, the adverse effects typical of opioids (36). In addition, dextropropoxiphen may cause respiratory arrest and/or lethal ventricular arrhythmias when combined with alcohol or taken as an overdose (36).

Paracetamol (acetaminophenol) is another frequently used antipyretic and analgesic agent, which differs from most NSAIDs in that it is a weak anti-inflammatory agent and does not produce the typical side effects related to COX-1 inhibition (11, 22). Although acetaminophen was introduced into clinical medicine more than a century ago, its mechanism of action is still unknown. A selective inhibition of prostaglandin synthesis in brain, consistent with a central site of action of acetaminophen (3, 29), has been proposed (19). However, acetaminophen is a very weak inhibitor of isolated COX-1 and COX-2 (FIG. 1), and there are now clear indications that the analgesic effects of acetaminophen involve molecular targets distinct from COX (1, 21, 32). A serious drawback of acetaminophen is its well-known toxic effects on liver and kidney, and liver necrosis is a feared complication in patients intoxicated with acetaminophen (11, 22). Thus, many of the existing analgesic, antipyretic and anti-inflammatory drugs are associated with serious side effects, why there is a need for more effective and less toxic drugs.

### SUMMARY OF THE INVENTION

Much interest has been focused on vanilloid and cannabinoid receptors as drug targets for treatment of pain and inflammation (35, 37). Both vanilloid and cannabinoid receptors are present in the pain and thermoregulatory pathways and mediate analgesia and hypothermia, and they also display an overlap in ligand recognition properties (34, 35, 37, 38). Others and we have recently reported that the fatty acid amide AM404 is a potent activator of rat (FIG. 2) and human vanilloid receptors (34, 47). AM404 is also a ligand at cannabinoid receptors and an inhibitor of the anandamide transporter, the inhibition of which leads to increased levels of endogenous cannabinoids (6, 38). As shown here, AM404 and similar fatty acid amides, including, inhibit both COX-1 and COX-2 (FIG. 1). Thus, it is not surprising that AM404 has anti-nociceptive properties and potentates the analgesic effect of anandamide in the mouse hot plate test (6, 10). Arachidonoyldopamine and oleyl vanillylamide (olvanil), other members of an increasing group of fatty acid amides acting on both vanilloid receptors and the endogenous cannabinoid system (38), have analgesic and anti-inflammatory effects and influence body temperature in a variety of in vivo assays (8, 23).

The endogenous fatty acid amide anandamide (arachidonoylethanolamide), which is an agonist at cannabinoid (35) and vanilloid (48) receptors, is hydrolysed to arachidonic acid and ethanolamine by a fatty acid amide hydrolase (FAA) (12, 13). This enzyme may also act in the reverse direction, causing synthesis of anandamide from arachidonic acid and ethanolamine (13). The structures of acetaminophen and AM404 differ only with regard to the length of the hydrocarbon chain. We have shown that acetaminophen, following deacetylation to its primary amine p-aminophenol, is conjugated with arachidonic acid to form AM404 (FIGS. 2, 4 and 5). Other primary amines, such as dopamine, serotonine and methoxy-3-tyramine, are also conjugated with arachidonic acid to form their respective arachidonoylderivatives (FIG. 6). Our discovery of AM404 as a metabolite of acetaminophen produced locally in the central nervous system provides an explanation for the mechanism of action of this widely consumed analgesic and antipyretic agent.

Many fatty acid amides and esters, including anandamide, AM404 and arachidonoyldopamine, are ligands at vanilloid and cannabinoid receptors (se references below). As shown here, 1-arachidonoylglycerol, 2-arachidonoylglycerol, arachidonoyl-3-methoxytyramine and arachidonoyltyramine are also activators of vanilloid receptors on perivascular sensory nerves (FIG. 7). Based on our discovery of a biochemical pathway for synthesis of fatty acid amides and existing knowledge of structure-activity relationships of vanilloid and cannabinoid receptor agonists (8, 14, 15, 18, 23-28, 30, 33, 34, 38-42, 46) and of the mechanisms behind the toxic effects of acetaminophen (4, 5, 7, 16, 20, 43, 44), we have designed compounds of low molecular weight and simple chemical structures, which are more effective and less toxic than acetaminophen in the treatment of fever, pain and inflammation.

These compounds can be represented by the general formula X—Y, in which X is an aromatic entity or a saturated or unsaturated cycloalkyl group, having the general formulas (I, II)

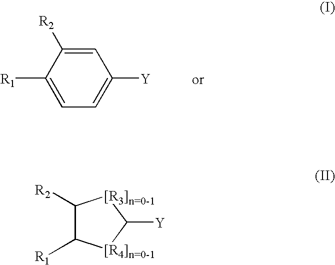

or a non-cyclic, straight or branched alkyl group, having the general formulas (III, IV)

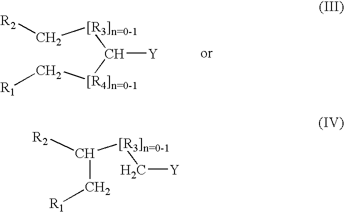

wherein R₁ and R₂ can be independently selected from hydrogen (—H), methyl (—CH₃), hydroxyl (—OH), hydroxymethyl (—CH₂OH), hydroxyethyl (—C₂H₅OH), —C₁₋₃-alkoxy, methoxymethyl (—CH₂OCH₃), methoxyethyl (—C₂H₅OCH₃), hydroxymethoxy (—OCH₂OH), hydroxyethoxy (—OC₂H₄OH), methoxymethoxy (—OCH₂OCH₃), methoxyethoxy (—OC₂H₄OCH₃), thiol (—SH), thiomethyl (—CH₂SH), thioethyl (—C₂H₅SH), methylthio (—SCH₃), ethylthio (—SC₂H₅), methylthiomethyl (—CH₂SCH₃), methylthioethyl (—C₂H₅SCH₃) nitro (—NO₂), aminomethoxy (—OCH₂NH₂), aminoethoxy (—OC₂H₅NH₂) and halogen (—Cl, —F, —Br or —I), preferably hydroxy, methoxy, ethoxy, aminomethoxy, aminoethoxy, chlorine and nitro; and whereby any hydroxy group of R₁ and R₂ may be protected by a metabolically deprotectable protecting group to provide —OH in situ (9); and

wherein R₃ can be —CR₅— or —CR₅CH— when X is an unsaturated cycloalkyl and —CHR₅— or —CHR₅CH₂— when X is a saturated cycloalkyl or a non-cyclic, straight or branched alkyl and R₄ can be —CR₆— or —CR₆CH— when X is an unsaturated cycloalkyl and —CHR₆— or —CHR₆CH₂— when X is a saturated cycloalkyl or a non-cyclic, straight or branched alkyl, wherein R₅ and R₆ can be independently selected from hydrogen (—H), methyl (—CH₃), ethyl (—C₂H₅), isopropyl (—C₃H₇) and halogen (—Cl, —F, —Br or —I); and

in which Y can be a primary amine (—R₇NH₂), hydroxyalkyl (—R₇OH) or thiolalkyl (—R₇SH), or —R₇NHC(O)R₈, —R₇NH(S)R₈, —R₇OC(O)R₈, —R₇OC(S)R₈, —R₇SC(O)R₈ or —R₇SC(S)R₈, wherein R₇ can be [CH₂]ₙ₌0-6, and R₈ can a straight or branched hydrocarbon chain (C₁₋₁₂), optionally substituted with a halogen (—F, —Cl, —Br or —I), —F₃, amine (—NH₂), hydroxy (—OH) or methoxy; and whereby any hydroxy group in Y may protected by a metabolically deprotectable protecting group to provide —OH in situ (9); and

with the proviso that R₁ and R₂ are not both hydrogen when X is a benzyl, the compound is not acetaminophen, phenactitin, acetamino-(3-hydroxy)-benzene, acetamino-(3-C₁₋₃-alkoxy)-benzene, acetamino-(3-hydroxy)-benzene, N-(3,4-dihydroxy-phenyl)methyl-C₅₋₁₁-alkylamide, N-((3-methoxy-4-hydroxyphenyl)methyl-C₅₋₁₁-alkylamide, N-(4-hydroxyphenyl)methyl-C₁₋₁₂-alkylamide, N-(4-(2-aminoethoxy)-phenyl)methyl-C₁₋₁₂-alkylamide, N-(3,4-dihydroxyphenyl)methyl-C₁₋₁₂-alkylamide, N-(3-methoxy-4-hydroxyphenyl)methyl-C₁₋₁₂-alkylamide, N-(3-hydroxy-4-(2-aminoethoxy)-phenyl)methyl-C₁₋₁₂-alkylamide, N-(3-methoxy-4-(2-aminoethoxy)-phenyl)methyl-C₁₋₁₂-alkylamide, acetamino-(3-C₁₋₃-alkylthio-4-C₁₋₃-alkoxy)-benzene, acetamino-(3-thiol-4-C₁₋₃-alkoxy)-benzene, acetamino-(3-C₁₋₃-alkylthio-4-hydroxy)-benzene or acetamino-(3-thiol-4-hydroxy)-benzene.

The general formulas III and VI are based on our observation that the ester compounds 1-arachidonoylglycerol and 2-arachidonoylglycerol are able to activate vanilloid receptors on sensory nerves (FIG. 7). These formulas can be regarded as modifications of the general formula I and III after opening of the ring structure. As demonstrated in example 2 to 4, compounds included in these formulas can be enzymatically conjugated with a fatty acid, preferably arachidonic acid. In order to undergo such a conjugation, each of the compounds having a R₈ fragment must first be hydrolysed to a primary amine, alcohol or thiol, which in turn is conjugated with the fatty acid via an amide, ester or thioester bond (FIG. 3). The acylderivatives formed in this reaction act as modulators of vanilloid receptors and/or various proteins of the endocannabinoid system, including cannabinoid receptors and the anandamide transporter.

The present invention further encompasses prodrugs of the present compounds, whereby such prodrugs have been provided with protecting groups, which metabolise into active compounds in the body. Metabolically removable protecting groups on hydroxyl groups consist of groups having ester or amide characteristics, including phenyl acetic acid derivatives (9).

The compounds of the present invention can be administered in the form of oral, rectal, injection or inhalator preparations. Oral compositions normally exist as tablets, granules, capsules (soft or hard) or powders, either coated or uncoated products. As coated products they may be merely enteric coated to provide for a more readily administered preparation, or as a sustained release coated composition, where the release of active compound will take place due to the dissolution of the coating, which dissolution is dependent on where in the gastro-intestinal tract one will have a release. Thus the release can be controlled as to place and time. It may also be advantageous to coat the active compound if this is subject to degradation, such as by gastric acid, in order then to have the compound to pass the stomach.

Tablets and capsules normally contain one dose of the active compound, i.e., the dose determined to fulfil the requirements of obtaining a therapeutically active level in serum or otherwise, either this is required once, twice or more times a day (24 hrs).

Rectal compositions are normally prepared as suppositories, where the active compound is dissolved or dispersed in a waxy compound or fat, having a melting temperature in the range of the body temperature, as to release the active compound when administered rectally.

Preparations for injection are commonly made for subcutaneous, intramuscular, intravenous, or intraperitoneal, epidural or spinal administration. Injection solutions are normally provided with an adjuvant to facilitate absorption of the active compound.

Preparations for inhalation are commonly present as powders which are administered either in pressurized containers with a dosing nozzle, or in an inhaler system where the powder is dosed in the system and then the patient is inhaling air through the apparatus to such degree that the powder becomes airborne and enters the respiratory tract, including the lungs. Inhalation preparations are normally used for inflammatory conditions in the respiratory tract including the lungs.

The compositions contain 0.5 to 99% by weight of active compound, and the remainder is different inert, non-therapeutically active compounds which facilitate administration, preparation such as granulation, tableting or storage. Such inert materials may, however, have a administratively positive effect.

Table 1 provides a list of non-exclusive, non-limiting applications provided by the method of treatment according to the invention.

Table 1. List of various symptoms, diseases and disorders that are treatable according to the methods of the invention.

Neurogenic Pain

Postherpetic neuralgia

Pain associated with diabetic neuropathy

Pain associated with chronic peripheral polyneuropathy

Stump pain after amputation

Postmastectomy pain syndrome

Pain associated with Gillain-Barrés disease

Horton's head ache

Nociceptive Pain

Osteoarthritis

Arthritis

Gout

Anaesthesia

Epidural and spinal anaesthesia

Local anaesthesia

Fever

Acute and chronic infections

Autoimmune and rheumatic diseases

Inflammatory bowel diseases

Inflammatory Diseases

Allergic and vasomotor (non-allergic) rhinitis

Nasopharyngeal adenoids

Eczema

Asthma

Urticaria

Psoriasis

Other Condition Related to Pain and Inflammation

Atherosclerosis

Cough

Itching of various aetiology

Urge incontinence

Protection against ulcer and mucosal damage in the gastro-intestinal tract

Wound healing

Neurodegenerative Disorders

Parkinson's disease

Alzheimer's

Huntington's disease

### LEGENDS TO FIGURES

FIG. 1. a, No effect of acetaminophen (AcAP) and p-aminophenol (AP) on COX-1 and COX-2 activity in isolated enzyme preparations (n=4-5). b, AM404 concentration-dependently inhibited both COX-1 and COX-2 activity. Indomethacin (10 μM) and the COX-2 selective inhibitor NS-398 (10 μM) almost abolished COX-1 (6±0.4%, n=4) and COX-2 (11±2%, n=6) activity, respectively (not shown). COX activity was measured as prostaglandin formation in the presence of 10 μM arachidonic acid.

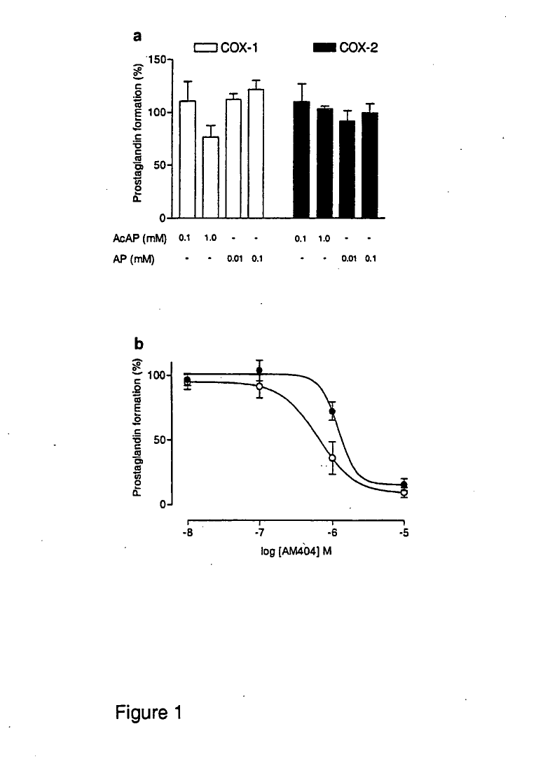

FIG. 2. Acetaminophen and p-aminophenol, in contrast to AM404, do not act on native vanilloid receptors in rat isolated mesenteric arteries. a, Representative traces showing no response to acetaminophen (AcAP) or p-aminophenol (AP) in arterial segments contracted with phenylephrine (n=5). Capsaicin (CAP) always relaxed these arteries. Dashed line indicates the basal tension level before addition of drugs. b, Concentration-response curves for capsaicin in arterial segments contracted with phenylephrine after treatment with 1 mM acetaminophen (triangles), 100 μm p-aminophenol (squares) or vehicle (circles) for 30 min (n=5). c, AM404 is a potent vasodilator (open circles) of arterial segments contracted with phenylephrine (n=11). The action of AM404 is inhibited by the competitive vanilloid receptor antagonist capsazepine (3 μM; filled circles; n=5) and the non-competitive vanilloid receptor antagonist ruthenium red (1 μM; diamonds; n=4). AM404 was unable to relax arteries pre-treated with capsaicin (1 μM) for 30 min (n=4; not shown) to cause vanilloid receptor desensitisation and/or depletion of sensory neuropeptides (48). Broken line (triangles) shows the relaxant effect of “endogenous” AM404 from rat homogenates incubated with p-aminophenol (mean of 4 arterial segments from the same rat). “Endogenous” AM404 was purified using LC and quantified by LC/MS-MS as described. Tension traces show relaxant responses to increasing concentrations of exogenous (upper trace) and “endogenous” (lower trace) AM404.

FIG. 3. Acetaminophen is metabolised to the primary amine p-aminophenol, which is further conjugated with arachidonic acid to form the bioactive fatty acid amide N-(4-hydroxyphenyl)-5,8,11,14-eicosatetraenamide (AM404).

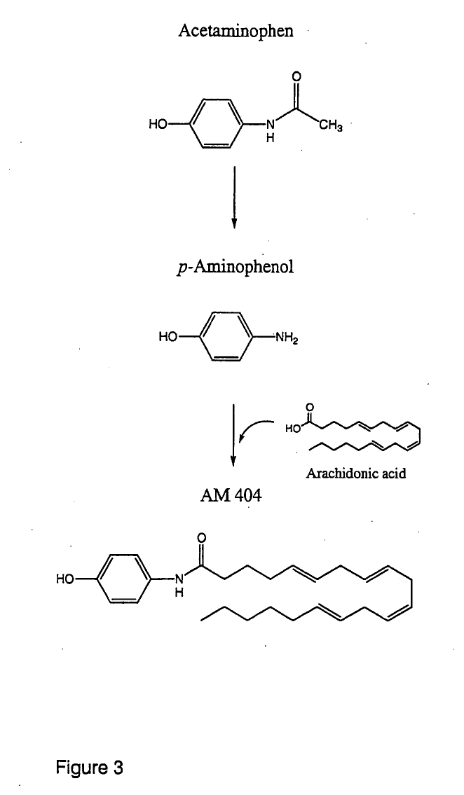

FIG. 4. Formation of AM404 and p-aminophenol in rat after intraperitoneal injection of acetaminophen (30-300 mg kg⁻¹) and its inhibition by PMSF (10 mg kg⁻¹). a,b, Representative chromatograms of samples obtained from rat brains showing (a) the presence of AM404 (23.4 pmol g⁻¹) in an animal treated with acetaminophen and (b) no AM404 in an animal injected with vehicle. The tandem mass spectrometer was operated to select the protonated molecular ion of AM404 at m/z 396.1 in the first quadruple mass separator, while the mass fragment at 109.8 after fragmentation in the collision cell (corresponding to the protonated p-aminophenol fragment) was selected by the second quadruple. c,d, Identification of AM404 and p-aminophenol in various tissues obtained from rats after exposure to acetaminophen or vehicle for 20 min in vivo (n=4-5; *P<0.016 compared to vehicle). e,f, Quantification of AM404 and p-aminophenol in brain after administration of different doses of acetaminophen (n=6-10). g, PMSF abolishes the formation of AM404 but only partly inhibits the formation of p-aminophenol in brain after administration of acetaminophen (n=5).

FIG. 5. The formation of AM404 in rat brain homogenates is dependent on p-aminophenol and is sensitive to the enzyme inhibitor PMSF. a, p-Aminophenol (10 μM; circles), but neither acetaminophen (100 μM; triangles) nor vehicle (not shown), causes a production of AM404 in brain homogenates (n=4). b, Formation of p-aminophenol from acetaminophen (100 μM) was detected in liver (circles), but not in brain (triangles) homogenates (n=4). No p-aminophenol could be detected in homogenates incubated with vehicle (n=4). c, d, Brain homogenates were incubated for 1 hour with either p-aminophenol plus arachidonic acid (each 100 μM) to generate AM404 or [²H₈]-anandamide (10 μM) to study its hydrolysis. Pre-incubation for 1 hour with PMSF inhibits (c) AM404 production and (d) [²H₈]-anandamide hydrolysis, measured as [²H₈]-arachidonic acid formation (n=4).

FIG. 6. The formation of arachidonoyldopamine and arachidonoylserotonin in rat brain homogenates is sensitive to the enzyme inhibitor PMSF. Homogenates were incubated with arachidonic acid (AA; 100 μM) alone or combined with (a) dopamine (DA; 100 μM) or (b) serotonin (5-HT; 100 μM) for 1 hour (n=3). The production of arachidonoyldopamine and arachidonoylserotonin was inhibited by PMSF (100 μM), but not by its ethanol vehicle (EtOH; 0.1%), added to homogenates 1 hour before the addition of arachidonic acid (in ethanol 0.1%) plus either dopamine or serotonin (n=3).

FIG. 7. Vanilloid receptor-dependent vasodilator action of different arachidonoyl derivatives in rat isolated mesenteric in arterial segments contracted with phenylephrine. Concentration-response curves for (a) 1-arachidonoylglycerol (1-AG) and (b) 2-arachidonoylglycerol (2-AG) in the absence (filled circles) and presence (filled triangles) of the competitive vanilloid receptor antagonist capsazepine (1 μM). The 3-methoxytyramine (circles), dopamine (triangles) and tyramine (squares) derivatives of arachidonic acid also induced concentration-dependent relaxation (c). None of the agonists elicited a relaxation after pre-treatment with 10 μM capsaicin for 30 min (open symbols) to cause vanilloid receptor desensitisation and/or depletion of sensory neuropeptides (48).

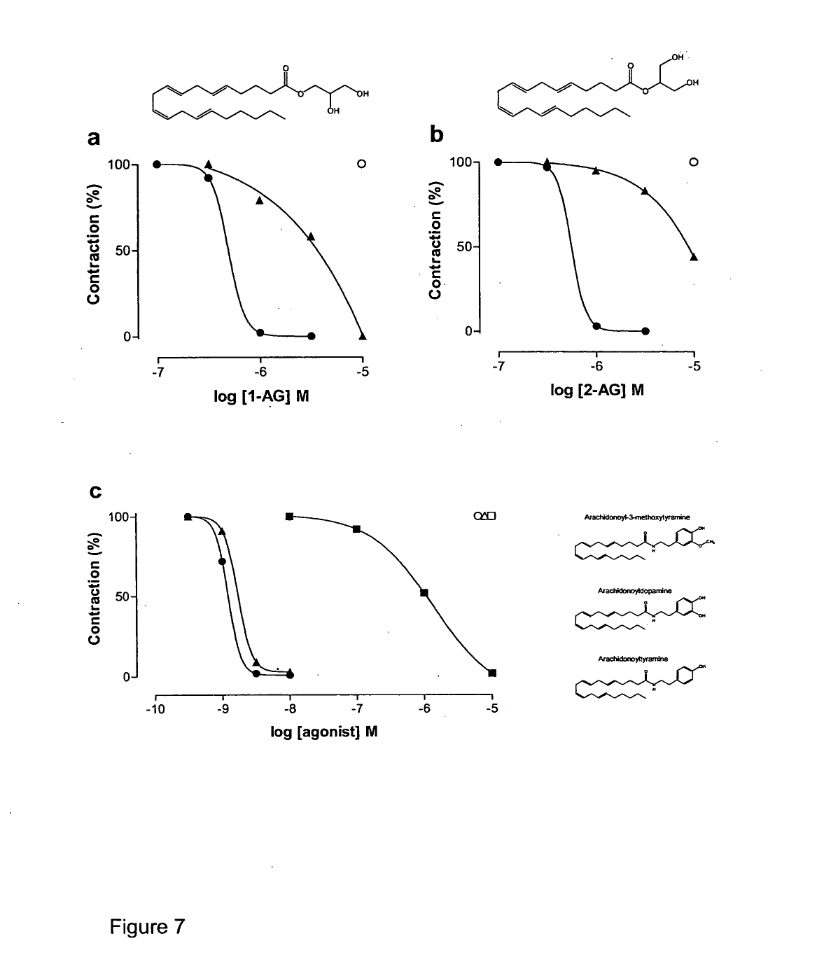

### EXPERIMENTAL PART

The invention will now be described in more detail with reference to specific examples of the invention, which are not intended to be, and should not be construed as, limiting the scope of the invention in any way.

Materials and Methods

Synthesis. The compounds of the present invention were synthesised in accordance with common practise, whereby the starting materials were synthesised as well, or were bought in bulk from common suppliers of organic chemicals.

In vivo experiments. Acetaminophen (300 mg k⁻¹) or vehicle (saline) at a volume of 2-3 ml was given to female Wistar-Hannover rats (200-300 g) by an intraperitoneal injection. Some rats were pretreated with PMSF (10 mg kg⁻¹) or vehicle (saline:PEG 6000; 1:10 w/w) given subcutaneously (2-3 ml) 20 min before administration of acetaminophen. Approximately 20 min after injection of acetaminophen, the animals were killed to collect brain, liver, spinal cord and arterial blood. The tissues were homogenised in a Tris buffer (10 mM, pH 7.6) containing EDTA (1 mM). PMSF (0.1 mM) and ascorbic acid (0.3 mM) were also present in the Tris buffer and added to the blood samples to prevent degradation of fatty acid amides and p-aminophenol, respectively. Aliquots (200 μl) of blood and homogenates were precipitated with 1 ml ice-cold acetone, containing 1 μM [²H₈]-labelled anandamide as internal standard. The samples were kept on ice until the acetone phase was evaporated in vacuo.

Tissue homogenate experiments. The brain, liver, spinal cord and dorsal root ganglia from female Wistar-Hannover rats (250 g) were homogenised in a Tris buffer (10 mM, pH 7.6), containing EDTA (1 mM), to give 90-330 mg tissue ml⁻¹. We carried out experiments in aliquots of 200 μl homogenate at 37° C. as further explained in the text. The reactions were stopped by adding 1 ml ice-cold acetone containing 1 μM [²H₈]-anandamide. The samples were kept on ice until the acetone phase was evaporated in vacuo.

Quantitative analyses. The extraction residues were reconstituted in 100 μl methanol except for p-aminophenol, for which 100 μl 0.5% acetic acid was used. The quantitative analyses were performed using a Perkin Elmer 200 liquid chromatography system with autosampler (Applied Biosystems), coupled to an API 3000 tandem mass spectrometer (Applied Biosystems/MDS-SCIEX). All mobile phases were water-methanol gradients, containing 0.5% acetic acid, and the flow rate was 200 μl min⁻¹ except for arachidonic acid where it was 400 μl min⁻¹.

AM404, arachidonoyldopamine, arachidonoylserotonin and anandamide. Sample aliquots of 5 μl were injected on a Genesis C₈ column (20×2.1 mm; Jones). Initially, the mobile flow was 25% water for 5.5 min. Then a linear gradient to 100% methanol was applied in 0.2 min and the mobile phase was kept at 100% methanol for 2.3 min, after which the column was reconditioned in 25% water for 2 min. The electrospray interface was operating in the positive ion mode at 370° C., the ion spray voltage was 4500 volts and the declustering potential was 40 volts. M/z 396.1/109.8 with a collision energy of 27 volts was used for the AM404 determinations. M/z 440.2/153.5 with a collision energy of 25 volts, m/z 463.2/159.6 with a collision energy of 39 volts and m/z 348.2/61.6 with a collision energy of 35 volts were used for arachidonoyldopamine, arachidonoylserotonin and native anandamide, respectively. M/z 356.4/62.2 with a collision energy of 35 volts was used for the internal standard [²H₈]-labelled anandamide.

p-Aminophenol. Sample aliquots of 2 μl were injected on a Genesis phenyl column (150×2.1 mm; Jones). The mobile flow was initially 97% water for 2 min. Then a linear gradient to 100% methanol was applied in 1 min and the mobile phase was kept at 100% methanol for 2 min, after which the column was reconditioned in 97% water for 3 min. The electrospray ion source was set at 450° C. and used in the positive ion mode. The ion spray voltage and declustering potential were set to 4500 volts and 55 volts, respectively. M/z 109.9/64.6 with a collision energy of 31 volts was used for the quantitative determinations.

[²H₈]-Arachidonic acid. Sample aliquots of 5 μl were injected on a Genesis C₁₈ column (50×2.1 mm; Jones). The HPLC was operated isocratically at 20% water and 80% methanol. The electrospray ion source was operating in the negative ion mode at 370° C., the ion spray voltage was −3000 volts and the declustering potential was −120 volts. M/z 310.8/267.0 with a collision energy of −22 volts was used for the quantitative determinations.

COX-1 and COX-2 assays. COX-1 and COX-2 activity was determined in the presence of 10 μM arachidonic acid using a COX (ovine) inhibitor screening assay (Cayman). Drugs were incubated with the enzyme preparation 8 min before application of arachidonic acid. Prostaglandin formation was used as a measure of COX activity and quantified via enzyme immunoassay (EIA).

Recording of tension. Experiments were performed on mesenteric arteries from female Wistar-Hannover rats (250 g) as described (48). Briefly, the arteries were cut into ring segments and mounted in tissue baths, containing aerated physiological salt solution (5% CO₂ and 95% O₂; 37° C.; pH 7.4). Experiments carried out in the presence of N^{G}-nitro-L-arginine (0.3 mM) and indomethacin (10 μM) to eliminate any contribution of nitric oxide and cyclo-oxygenase products, respectively. We studied relaxant responses in preparations contracted with phenylephrine. When stable contractions were obtained, substances were added cumulatively to determine concentration-response relationships.

Calculations and statistics. Data are presented as means ±S.E.M. (vertical lines in figures), and n indicates the number of animals unless otherwise stated. GraphPad Prism 3.0 software was used for curve fitting (non-linear regressions) and calculations of pEC₅₀ values. Mann-Whitney U-test or Student's t-test on log transformed values was used for statistical analysis. Statistical significance was accepted when P<0.05.

Drugs. Acetaminophen, p-aminophenol, N^{G}-nitro-L-arginine, ascorbic acid, dopamine, phenylephrine, PMSF, ruthenium red, serotonin (all from Sigma) and indomethacin (Confortid, Dumex) were dissolved in and diluted with distilled water. AM404, capsaicin, capsazepine (all from Tocris); [²H₈]-anandamide, [²H₈]-arachidonic acid, arachidonoylserotonin, NS-398 (all from Cayman); anandamide (Biomol); arachidonic acid (Sigma); arachidonoyl-dopamine, arachidonoyl-3-methoxytyramine and arachidonoyltyramine (Syntelec) were all dissolved in and diluted with ethanol. DMSO substituted ethanol as a solvent in the COX assays. The batch of acetaminophen contained no or less than 0.001% (w/w) of p-amino-phenol, as determined by LC/MS-MS.

### EXAMPLE 1

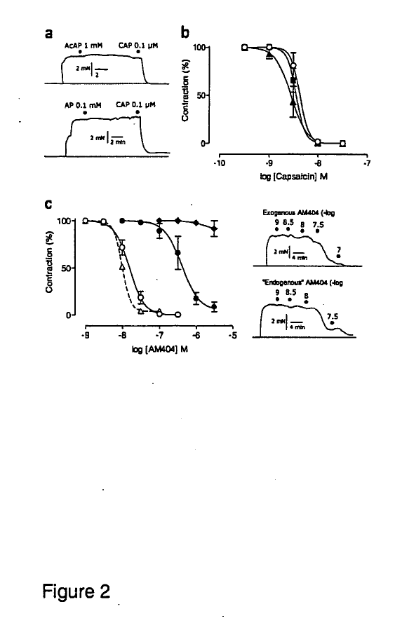

The vasodilator effects of AM404, capsaicin, acetaminophen and aminophenol in isolated segments of rat mesenteric arteries, a well-defined and very sensitive bioassay system of vanilloid receptor active drugs (48), were also examined. As shown in FIG. 2, AM404 and capsaicin are potent agonists at vanilloid receptors on vasodilator sensory nerves (AM404: pEC₅₀=7.80±0.01, n=11; Capsaicin: pEC₅₀=8.36±0.05, n=5). Acetaminophen and p-aminophenol (in the presence of ascorbic acid to prevent its decomposition) neither induced vasorelaxation per se nor inhibited the effect of capsaicin in this bioassay system, indicating lack of agonist and antagonist actions on vanilloid receptors (FIG. 2).

### EXAMPLE 2

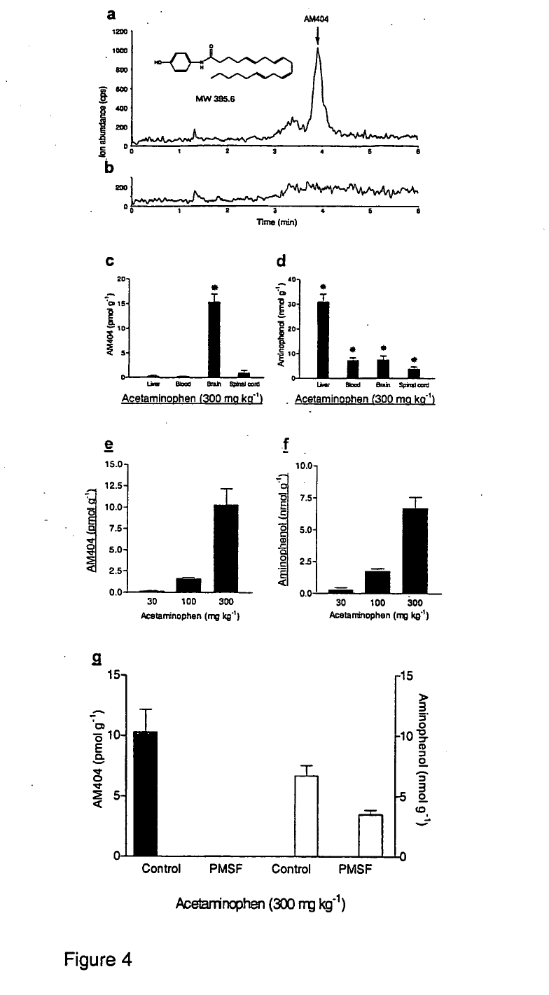

Since the structures of acetaminophen and AM404 differs only with regard to the length of the hydrocarbon chain, we hypothesised that acetaminophen, following deacetylation to its metabolite p-aminophenol (31), is conjugated with arachidonic acid to form AM404 (FIG. 4). To test this proposal, we measured the levels of AM404 and p-aminophenol in various tissues of rat 20 min after intraperitoneal injection of acetaminophen at a commonly used dose (300 mg/kg), which produces a robust analgesic effect in rodents (17, 21, 45). In all five animals exposed to acetaminophen, substantial levels of AM404 were observed in brain (15±1.6 pmol g⁻¹). AM404 could also be detected in the spinal cord in two out of five animals, but was absent in liver and blood (FIG. 4). p-Aminophenol was present in all tissues (FIG. 4), of which the liver contained the highest levels (31±3.2 mmol g⁻¹). Pre-treatment with the FAAH inhibitor PMSF abolished the formation of AM404 in brain, while the p-aminophenol content was reduced by 48% (FIG. 4). AM404 and p-aminophenol could not be detected in vehicle-treated animals (n=4), whereas the levels of anandamide in the same samples of brain and spinal cord were 10±0.5 pmol g⁻¹ and 7.0±0.6 pmol g⁻¹, respectively (n=4).

### EXAMPLE 3

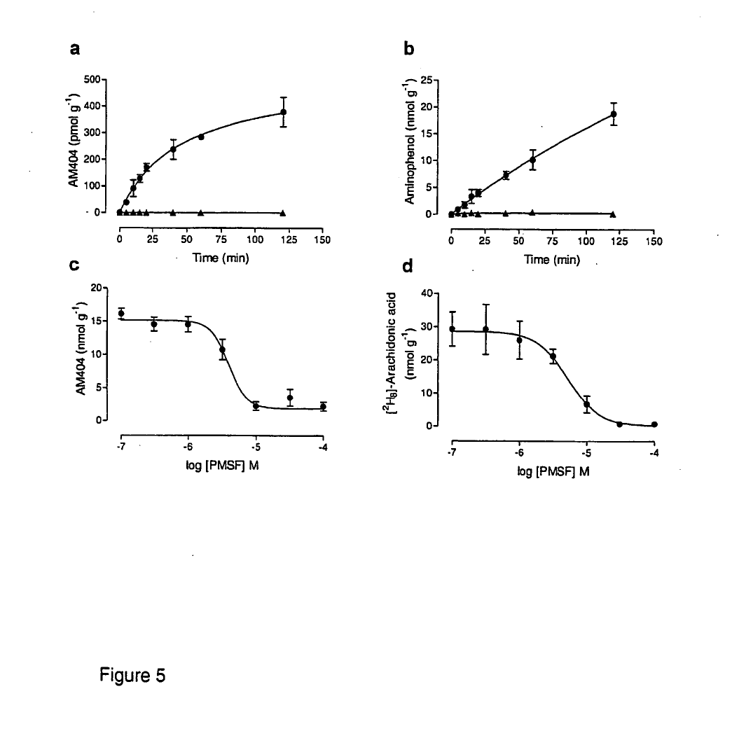

To further characterise the formation of AM404 and p-aminophenol, homogenates of rat brain and liver were incubated with p-aminophenol and acetaminophen for various time periods. Exposure to p-aminophenol (10 μM) produced a time-dependent formation of AM404 in brain homogenates, whereas incubation with acetaminophen (100 μM) did not result in any detectable levels of AM404 (FIG. 5a). Likewise, p-aminophenol could not be measured in brain homogenates incubated with acetaminophen (FIG. 5b). However, we cannot exclude that small but relevant amounts of p-aminophenol is produced in brain, since significant amounts of AM404 (14±2.6 pmol g⁻¹, n=4) was measured in brain homogenates incubated with a ten times higher concentration of acetaminophen (1 mM). Indeed, this amount of AM404 would correspond to a p-aminophenol concentration below the detection limit of the assay. Substantial amounts of p-aminophenol could, however, be detected in liver homogenates incubated with acetaminophen (FIG. 5b).

Since primary sensory nerves of dorsal root ganglia and connecting neurones in the spinal cord are potential cellular targets for analgesic drugs acting, directly or indirectly, on vanilloid and cannabinoid receptors (2, 35, 37), it was considered of interest to see if AM404 could be formed in these tissues. Indeed, formation of AM404 was demonstrated in homogenates of rat spinal cord (24±2.2 pmol g⁻¹, n=4) and dorsal root ganglia (10±1.8 pmol g⁻¹, n=4) incubated with p-aminophenol (10 μM) for 1 hour. The level of AM404 was enhanced 6-fold when the homogenates were supplemented with arachidonic acid (100 μM) and the p-aminophenol concentration was increased 10 times (spinal cord: 161±20 pmol g⁻¹ n=4; dorsal root ganglia: 62±1.5 pmol g⁻¹, duplicate measurements of pooled homogenates from four animals).

As further shown in rat brain homogenates, AM404 is formed via an enzyme-dependent process. First, AM404 could not be detected in homogenates boiled for 10 min before incubated with p-aminophenol (100 μM) and arachidonic acid (100 μM) for 1 hour (n=4). Second, phenyl-methyl-sulphonylfluoride (PMSF), a broad-spectrum protease, esterase and amidase inhibitor (13), concentration-dependently inhibited the formation of AM404 with a pEC₅₀ value of 5.41±0.03 (n=4; FIG. 5c). This compound also inhibited the hydrolysis of anandamide with a similar pEC₅₀ value (5.28±0.07, n=4; FIG. 5d).

### EXAMPLE 4

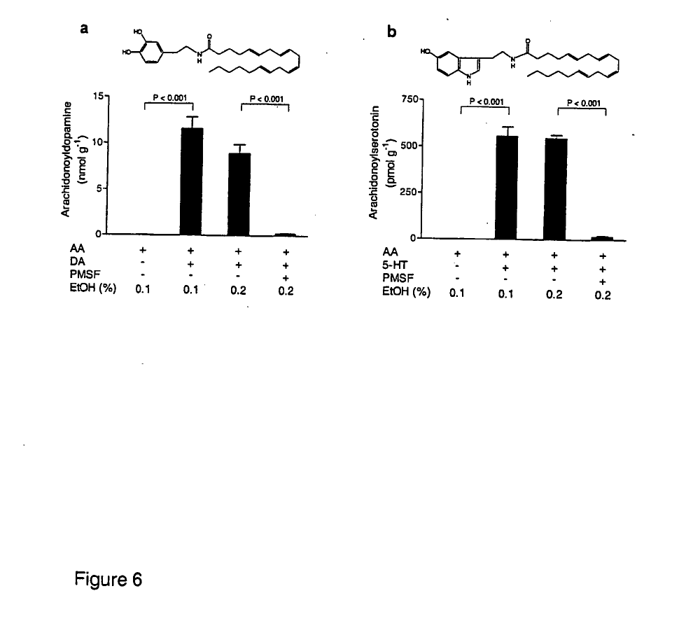

We also tested whether the endogenous monoamines dopamine and serotonin could be converted to their respective arachidonoylderivatives. Incubation of brain homogenates with dopamine or serotonin led to the production of substantial amounts of arachidonoyldopamine and arachidonoylserotonin (FIG. 6). The enzyme inhibitor PMSF almost abolished the formation of these fatty acid amides (FIG. 6). Thus, not only p-aminophenol, but also endogenous monoamines are enzymatically conjugated with arachidonic acid to form bioactive fatty acid amides.

### REFERENCES

1. Abbott, F. V., and K. G. C. Hellemans. Phenacetin, acetaminophen and dipyrone: analgesic and rewarding effects. Behavioural Brain Research 112: 177-186, 2000.

2. Ahluwalia, J., L. Urban, M. Capogna, S. Bevan, and I. Nagy. Cannabinoid 1 receptors are expressed in nociceptive primary sensory neurons. Neuroscience 100: 685-688, 2000.

3. Bannwarth, B., F. Demotes-Mainard, T. Schaeverbeke, L. Labat, and J. Dehais. Central analgesic effects of aspirin-like drugs. Fundamentals in Clinical Pharmacology 9: 1-7, 1995.

4. Barnard, S., D. F. Kelly, R. C. Storr, and B. K. Park. The effect of fluorine substitution on the hepatotoxicity and metabolism of paracetamol in the mouse. Biochemical Pharmacology 46: 841-849, 1993.

5. Barnard, S., R. C. Storr, P. M. O'Neill, and B. K. Park. The effect of fluorine substitution on the physicochemical properties and the analgesic activity of paracetamol. Journal of Pharmcy and Pharmacology 45: 736-744, 1993.

6. Beltramo, M., N. Stella, A. Calignano, S. Y. Lin, A. Makriyannis, and D. Piomelli. Functional role of high-affinity anandamide transport, as revealed by selective inhibition. Science 277: 1094-1097, 1997.

7. Bessems, J. G., H. D. Gaisser, J. M. Te Koppele, W. P. Van Bennekom, J. N. Commandeur, and N. P. Vermeulen. 3,5-Disubstituted analogues of paracetamol. Synthesis, analgesic activity and cytotoxicity. Chemico-Biological Interactions 98: 237-250, 1995.

8. Bisogno, T., D. Melck, M. Y. Bobrov, N. M. Gretskaya, V. V. Bezuglow, L. D. Petrocellis, and V. D. Marzo. N-acyl-dopamines: novel synthetic CB₁ cannabinoid-receptor ligands and inhibitors of anandamide inactivation with cannabimimetic activity in vitro and in vivo. Biochemical Journal 351: 817-824, 2000.

9. Bundgaard, H. Design of prodrugs. New York: Elsevier Science Publisher, 1985. p. 372.

10. Burstein, S. H., R. G. Rossetti, B. Yagen, and R. B. Zurier. Oxidative metabolism of anandamide. Prostaglandins & other Lipid Mediators 61: 29-41, 2000.

11. Clissold, S. P. Paracetamol and phenacetin. Drugs 32: 46-59, 1986.

12. Cravatt, B. F., D. K. Giang, S. P. Mayfield, D. L. Boger, R. A. Lerner, and N. B. Gilula. Molecular characterization of an enzyme that degrades neuromodulatory fatty-acid amides. Nature 384: 83-87, 1996.

13. Deutsch, D. G., and S. A. Chin. Enzymatic synthesis and degradation of anandamide, a cannabinoid receptor agonist. Biochemical Pharmacology 46: 791-796, 1993.

14. Di Marzo, V., T. Bisogno, L. De Petrocellis, I. Brandi, R. G. Jefferson, R. L. Winckler, J. B. Davis, O. Dasse, A. Mahadevan, R. K. Razdan, and B. R. Martin. Highly Selective CB(1) Cannabinoid Receptor Ligands and Novel CB(1)/VR(1) Vanilloid Receptor “Hybrid” Ligands. Biochemical and Biophysical Research Communications 281: 444-451, 2001.

15. Di Marzo, V., T. Bisogno, D. Melck, R. Ross, H. Brockie, L. Stevenson, R. G. Pertwee, and L. De Petrocellis. Interactions between synthetic vanilloids and the endogenous cannabinoid system. FEBS Letters 436: 449-454, 1998.

16. Fernando, C. R., I. C. Calder, and K. N. Ham. Studies on the mechanism of toxicity of acetaminophen. Synthesis and reactions of N-acetyl-2,6-dimethyl- and N-acetyl-3,5-dimethyl-p-benzoquinone imines. Journal of Medicinal Chemistry 23: 1153-1159, 1980.

17. Ferreira, S. H., B. B. Lorenzetti, and F. M. Correa. Central and peripheral antialgesic action of aspirin-like drugs. European Journal Pharmacology 53: 39-48, 1978.

18. Fichera, M., G. Cruciani, A. Bianchi, and G. Musumarra. A 3D-QSAR study on the structural requirements for binding to CB(1) and CB(2) cannabinoid receptors. Journal of Medicinal Chemistry 43: 2300-2309, 2000.

19. Flower, R. J., and J. R. Vane. Inhibition of prostaglandin synthetase in brain explains the anti-pyretic activity of paracetamol (4-acetamidophenol). Nature 240: 410-411, 1972.

20. Harvison, P. J., A. Forte, and S. D. Nelson. Comparative toxicities and analgesic activities of three monomethylated analogues of acetaminophen. Journal of Medicinal Chemistry 29: 1737-1743, 1986.

21. Hunskaar, S., and K. Hole. The formalin test in mice: dissociation between inflammatory and non-inflammatory pain. Pain 30: 103-114, 1987.

22. Insel, P. A. Analgesic-antipyretic and antiinflammatory agents and drugs employed in the treatment of gout. In: Goodman & Gilman's The Pharmacological Basis of Therapeutics (Ninth Edition ed.), edited by J. G. Hardman, A. G. Gilman and L. E. Limbird The McGraw-Hill Companies, Inc, 1996, p. 617-657.

23. Janusz, J. M., B. L. Buckwalter, P. A. Young, T. R. LaHann, R. W. Farmer, G. B. Casting, M. E. Loomans, G. A. Kerckaert, C. S. Maddin, E. F. Berman, R. L. Bohne, T. L. Cupps, and J. R. Milstein. Vanilloids. 1. Analogs of capsaicin with antinociceptive and antiinflammatory activity. Journal of Medical Chemistry 36: 2595-2604, 1993.

24. Jarrahian, A., S. Manna, W. S. Edgemond, W. B. Campbell, and C. J. Hillard. Structure-activity relationships among N-arachidonylethanolamine (Anandamide) head group analogues for the anandamide transporter. Journal of Neurochemistry 74: 2597-2606, 2000.

25. Khanolkar, A. D., and A. Makriyannis. Structure-activity relationships of anandamide, an endogenous cannabinoid ligand. Life Sciences 65: 607-616, 1999.

26. Lang, W., C. Qin, S. Lin, A. D. Khanolkar, A. Goutopoulos, P. Fan, K. Abouzid, Z. Meng, D. Biegel, and A. Makriyannis. Substrate specificity and stereoselectivity of rat brain microsomal anandamide amidohydrolase. Journal of Medicinal Chemistry 42: 896-902, 1999.

27. Lee, J., J. Lee, J. Kim, S. Y. Kim, M. W. Chun, H. Cho, S. W. Hwang, U. Oh, Y. H. Park, V. E. Marquez, M. Beheshti, T. Szabo, and P. M. Blumberg. N-(3-Acyloxy-2-benzylpropyl)-N′-(4-hydroxy-3-methoxybenzyl) thiourea derivatives as potent vanilloid receptor agonists and analgesics. Bioorganics and Medicinal Chemistry 9: 19-32.

28. Lin, S., A. D. Khanolkar, P. Fan, A. Goutopoulos, C. Qin, D. Papahadjis, and A. Makriyannis. Novel analogues of arachidonylethanolamide (anandamide): affinities for the CB1 and CB2 cannabinoid receptors and metabolic stability. Journal of Medicinal Chemistry 41: 5353-5361, 1998.

29. McCormack, K. Non-steroidal anti-inflammatory drugs and spinal nociceptive processing. Pain 59: 9-43, 1994.

30. Melck, D., T. Bisogno, L. De Petrocellis, H. Chuang, D. Julius, M. Bifulco, and V. Di Marzo. Unsaturated long-chain N-acyl-vanillyl-amides (N-AVAMs): vanilloid receptor ligands that inhibit anandamide-facilitated transport and bind to CB1 cannabinoid receptors. Biochemical and Biophysical Research Communications 262: 275-284, 1999.

31. Newton, J. F., C. H. Kuo, M. W. Gemborys, G. H. Mudge, and J. B. Hook. Nephrotoxicity of p-aminophenol, a metabolite of acetaminophen, in the fischer 344 rat. Toxicology and Applied Pharmacology 65: 336-344, 1982.

32. Pelissier, T., A. Alloui, F. Caussade, C. Dubray, A. Cloarec, J. Lavarenne, and A. Eschalier. Paracetamol exerts a spinal antinociceptive effect involving an indirect interaction with 5-hydroxytryptamine₃ receptors: in vivo and in vitro evidence. Journal of Pharmacology and Experimental Therapeutics 278: 8-14, 1996.

33. Pertwee, R. G. Pharmacology of cannabinoid CB1 and CB2 receptors. Pharmacology and Therapeutics 74: 129-180, 1997.

34. Petrocellis, L. D., T. Bisogno, J. B. Davis, R. J. Pertwee, and V. D. Marzo. Overlap between the ligand recognition properties of the anandamide transporter and the VR1 vanilloid receptor: inhibitors of anandamide uptake with negligible capsaicin-like activity. Federation of European Biochemical Societies Letters 483: 52-56, 2000.

35. Piomelli, D., A. Giuffrida, A. Calignano, and F. R. d. Fonseca. The endocannabinoid system as a target for therapeutic drugs. Trends in Pharmacological Sciences 21: 218-224, 2000.

36. Reisine, T., and G. Pasternak. Opioid analgesics and antagonists. In: Goodman & Gilman's The Pharmacological Basis of Therapeutics (Ninth edition ed.), edited by J. G. Hardman, A. G. Gilman and L. E. Limbird The McGraw-Hill Companies, Inc, 1996, p. 521-555.

37. Szallasi, A., and P. M. Blumberg. Vanilloid (capsaicin) receptors and mechanisms. Pharmacological reviews 51: 159-211, 1999.

38. Szallasi, A., and V. D. Marzo. New perspectives on enigmatic vanilloid receptors. Trends in Neurological Sciences 23: 491-497, 2000.

39. Walpole, C. S., S. Bevan, G. Bovermann, J. J. Boelsterli, R. Breckenridge, J. W. Davies, G. A. Hughes, I. James, L. Oberer, J. Winter, and R. Wrigglesworth. The discovery of capsazepine, the first competitive antagonist of the sensory neuron excitants capsaicin and resiniferatoxin. Journal of Medical Chemistry 37: 1942-1954, 1994.

40. Walpole, C. S., R. Wrigglesworth, S. Bevan, E. A. Campbell, A. Dray, I. F. James, K. J. Masdin, M. N. Perkins, and J. Winter. Analogues of capsaicin with agonist activity as novel analgesic agents; structure-activity studies. 2. The amide bond “B-region”. Journal of Medical Chemistry 36: 2373-2380, 1993.

41. Walpole, C. S., R. Wrigglesworth, S. Bevan, E. A. Campbell, A. Dray, I. F. James, K. J. Masdin, M. N. Perkins, and J. Winter. Analogues of capsaicin with agonist activity as novel analgesic agents; structure-activity studies. 3. The hydrophobic side-chain “C-region”. Journal of Medical Chemistry 36: 2381-2389, 1993.

42. Walpole, C. S., R. Wrigglesworth, S. Bevan, E. A. Campbell, A. Dray, I. F. James, M. N. Perkins, D. J. Reid, and J. Winter. Analogues of capsaicin with agonist activity as novel analgesic agents; structure-activity studies. 1. The aromatic “A-region”. Journal of Medical Chemistry 36: 2362-2372, 1993.

43. van de Straat, R., J. de Vries, E. J. Groot, R. Zijl, and N. P. Vermeulen. Paracetamol, 3-monoalkyl- and 3,5-dialkyl derivatives: comparison of their hepatotoxicity in mice. Toxicology and Applied Pharmacology 89: 183-189, 1987.

44. van De Straat, R., J. De Vries, T. Kulkens, A. J. Debets, and N. P. Vermeulen. Paracetamol, 3-monoalkyl- and 3,5-dialkyl derivatives. Comparison of their microsomal cytochrome P-450 dependent oxidation and toxicity in freshly isolated hepatocytes. Biochemical Pharmacology 35: 3693-3699, 1986.

45. Vaz, Z. R., V. C. Filho, R. A. Yunes, and J. B. Calixto. Antinociceptive action of 2-(4-bromobenzoyl)-3-methyl-4,6-dimethoxy benzofuran, a novel xanthoxyline derivative on chemical and thermal models of nociception in mice. Journal of Pharmacology and Experimental Therapeutics 278: 304-312, 1996.

46. Wrigglesworth, R., C. S. Walpole, S. Bevan, E. A. Campbell, A. Dray, G. A. Hughes, I. James, K. J. Masdin, and J. Winter. Analogues of capsaicin with agonist activity as novel analgesic agents: structure-activity studies. 4. Potent, orally active analgesics. Journal of Medical Chemistry 39: 4942-4951, 1996.

47. Zygmunt, P. M., C. Huai-Hu, P. Movahed, D. Julius, and E. D. Högestätt. The anandamide transport inhibitor AM404 activates vanilloid receptors. European Journal of Pharmacology 396: 39-42, 2000.

48. Zygmunt, P. M., J. Petersson, D. A. Andersson, H. Chuang, M. Sorgard, V. Di Marzo, D. Julius, and E. D. Hogestatt. Vanilloid receptors on sensory nerves mediate the vasodilator action of anandamide. Nature 400: 452-457, 1999.

1. An analgesic, antipyretic and/or anti-inflammatory compound represented by the general formula X—Y, in which X is a benzyl group, a saturated or unsaturated cycloalkyl group (I, II) or a non-cyclic, straight or branched alkyl group (III, IV), having the general formulas 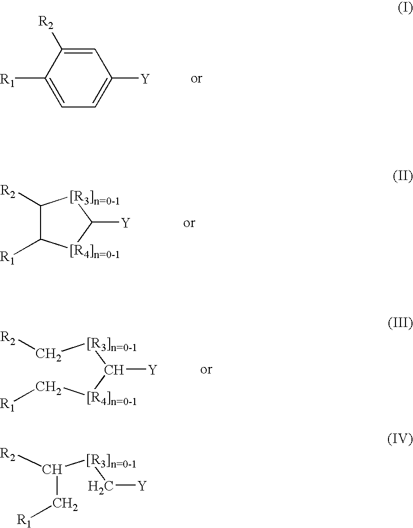
wherein R₁ and R₂ can be independently selected from hydrogen (—H), methyl (—CH₃), hydroxy (—OH), hydroxymethyl (—CH₂OH), 2-hydroxyethyl (—C₂H₅OH), -Ci.₃-alkoxy, methoxy-methyl (—CH₂OCH₃), methoxyethyl (—C₂H₅OCH3), hydroxymethoxy (—OCH₂OH), hydroxyethoxy (—OC₂H4OH), methoxymethoxy (—OCH₂OCH₃), ethoxymethoxy (—OC₂H4OCH₃), thiol (—SH), thiolmethyl (—CH₂SH), thiolethyl (—C₂H₅SH), methylthio (—SCH₃), ethylthio (—SC₂H₅), methylthiomethyl (—CH₂SCH₃), methylthioethyl (—C₂H₅SCH₃) nitro (—NO₂), aminomethoxy (—OCH2NH₂), aminoethoxy (—OC₂HsNH₂) and halon (—Cl, —F, —Br or —I); and whereby any hydroxy group of R₁ and R₂ may be protected by a metabolically deprotectable protecting group to provide —OH in situ; and
wherein R₃ can be —CR₅— or —CR₅CH— when X is an unsaturated cycloalkyl and —CHR₅— or —CHR₅CH₂— when X is a saturated cycloalkyl or a straight or branched alkyl and R4 can be —CR₆— or —CR₆CH— when X is an unsaturated cycloalkyl and —CHR₆— or —CHR₆CH₂— when X is a saturated cycloalkyl or a straight or branched alkyl, wherein R₅ and R₆ can be independently selected from hydrogen (—H), methyl (—CH₃), ethyl (—C₂H5), isopropyl (—C₃H₇) and halogen (—Cl, —F, —Br or —I); and
in which Y can be a primary amine (—R₇NH₂), hydroxyalkyl (—RvOH) or thioalkyl (—R₇SH), or —R₇NHC(0)R₈, —R₇NH(S)R₈, —R₇OC(0)R₈ —R₇OC(S)R₈, —R₇SC(O)R₈ or —R₇SC(S)R₈, wherein R₇ can be [CH₂]ₙ₌ₒ₋₆ and R₈ can be a straight or branched hydrocarbon chain (C₁₋₁₂), optionally substituted with a halogen (—F, —Cl, —Br or —I), —F₃, amine (—NH₂), hydroxy (—OH) or methoxy; and
with the proviso that R₁ and R₂ are not both hydrogen when X is benzyl, and that the compound is not acetaminophen, phenactitin, acetamino-(3-hydroxy)-benzene, acetamino-(3-C₁₋₃-alkoxy)-benzene, acetamino-(3-hydroxy)-benzene, N-((3,4-dihydroxy-phenyl)methyl-C₅₋₁₁-alkylamide, N-(3-methoxy-4-hydroxyphenyl)methyl-C₅₋₁₁-alkylamide, N-(4-hydroxyphenyl)methyl-C₁₋₁₂-alkylamide, N-(4-(2-aminoethoxy)-phenyl)methyl-C₁₋₁₂-alkylamide, N-(3,4-dihydroxy-phenyl)methyl-C₁₋₁₂-alkylamide, N-(3-methoxy-4-hydroxy-phenyl)methyl-C₁₋₁₂-alkylamide, N-(3-hydroxy-4-(2-aminoethoxy)-phenyl)methyl-C₁₋₁₂-alkylamide, N-(3-methoxy-4-(2-aminoethoxy)-phenyl)methyl-C₁₋₁₂-alkylamide, acetamino-(3-C₁₋₃-alkylthio-4-C₁₋₃-alkoxy)-benzene, acetamino-(3-thiol-4-C₁₋₃-alkoxy)-benzene, acetamino-(3-C₁₋₃-alkylthio-4-hydroxy)-benzeneoracetamino-(3-thiol-4-hydroxy)-benzene.

2. A compound according to claim 1, wherein Y is selected from —NH₂, —CH₂NH2, —CH₂CH₂NH₂, —NHCOR₈, —CH₂NHCOR₈ and —CH₂CH₂NHOR₈, wherein R₈ has the meaning given.

3. A compound according to claims 1 or 2, wherein R₈ is selected from —CH₃, —CF₃ and C₂₋₁₂-alkyl.

4. A compound according to claim 1, wherein R₁ and R₂ are independently selected from —OH, —OCH₃, —OCH₂CH₃, —OCH₂NH₂, —OCH₂CH₂NH₂, —Cl and —NH₂, and whereby R₁ (or R₂) is not —H when R₂ (or R₁) is —H or —OCH₃.

5. A compound according to claim 4, having the formula (IV) 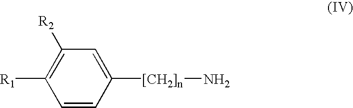
wherein n=0-2, and R₁ and R₂ have the meanings as given in claims 1-4.

6. A compound according to claim 4, having the formula (V) 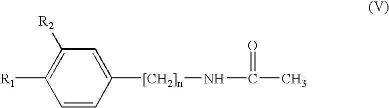
wherein n-0-2, and R₁ and R₂ have the meanings as given in claims 1-4.

7. A compound according to claim 4, having the formula (VI) 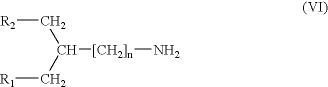
wherein n=0-4, and R₁ and R₂ have the meanings as given in claims 1-4.

8. A compound according to claim 4, having the formula (VII) 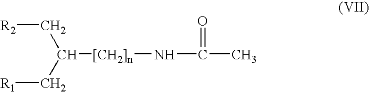
wherein n=0-4, and R₁ and R₂ have the meanings as given in claims 1-4.

9. A compound according to claim 4, having the formula (VIII) 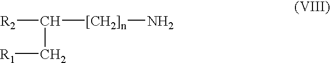
wherein n=0-4, and R₁ and R₂ have the meanings as given in claims 1-4.

10. A compound according to claim 4, having the formula (IX) 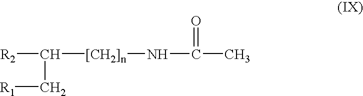
wherein n=0-4, and R₁ and R₂ have the meanings as given in claims 1-4.

11. A compound according to claim 1, wherein the fatty acid amide, ester or thioester is a derivative acting on the vanilloid receptor, the cyclo-oxygenases and/or the endocannabinoid system, including the cannabinoid receptors and the anandamide transporter.

12. A compound according to claim 1 for use as a medicament.

13. A pharmaceutical composition comprising a compound according to claim 1 as active ingredient together with a pharmaceutically acceptable adjuvant, diluent or carrier for the treatment of pain, fever and inflammations, optionally in combination with another analgesic, such as an NSAED or an opioid.

14. Use of a compound according to claim 1, and pharmaceutically acceptable salt thereof, for the manufacture of a medicament for treatment of pain.

15. Use of a compound according to claim 1, and pharmaceutically acceptable salt thereof, for the manufacture of a medicament for treatment of fever.

16. Use of a compound according to claim 1, and pharmaceutically acceptable salt thereof, for the manufacture of a medicament for treatment of inflammation.

17. A method for treatment of pain, fever and/or inflammation, wherein said method comprises administering a therapeutically effective amount of a compound according to claim 1.

18. The method of claim 17, wherein said administering comprises topical administration of a therapeutically effective amount by contacting skin or mucous membrane of a compound.

19. The method of claim 17, wherein said administering comprises oral administration of a therapeutically effective amount of a compound.

20. The method of claim 17, wherein said administering comprises administration of a therapeutically effective amount by injection locally, epidurally or spinally of a compound.
